(19) United States
(12) Patent Application Publication
(10) Pub. No.: US 2009/0054711 A1
(43) Pub. Date: Feb. 26, 2009

(54) Pyrolysis Systems, Methods of Use Thereof, and Methods of Bio-Oil Transformation

(76) Inventors: Tom Lawrence, Athens, GA (US); K.C. Das, Athens, GA (US)

Correspondence Address: THOMAS, KAYDEN, HORSTEMEYER & RISLEY, LLP, 600 GALLERIA PARKWAY, S.E., STE 1500, ATLANTA, GA, 30339-5994, US

(21) Appl. No.: 11/913,425

(22) PCT Filed: May 2, 2006
(86) PCT No.: PCT/US06/16687

§ 371 Date: May 28, 2008

Related U.S. Application Data
(60) Provisional application No. 60/677,720, filed on May 4, 2005.

(51) Int. Cl. C07C 1/00; C10G 1/00; C10G 9/36

(52) U.S. Cl. 585/240; 202/96

(57) ABSTRACT

Pyrolysis systems and methods of recovering bio-oil product of are disclosed. An illustrative embodiment of a pyrolysis system, among others, includes: a pyrolysis reactor that produces a first bio-oil stream; an injection spray system for receiving the first bio-oil stream, wherein the injection spray system is configured to spray a solvent liquid onto the first bio-oil stream to produce a second bio-oil stream, wherein the injection spray system is configured to control the removal of heat from the first bio-oil stream; and a post injection spray system that is configured to receive the second bio-oil stream.

### CROSS REFERENCE TO RELATED APPLICATION

The present application claims priority to and the benefit of U.S. provisional patent application Ser. No. 60/677,720, filed on May 4, 2005 and entitled “PYROLYSIS SYSTEMS AND METHODS OF USE THEREOF”, which is incorporated herein by reference in its entirety.

### TECHNICAL FIELD

The present disclosure is generally related to systems and methods for treating biomass and, more particularly, is related to systems and methods related to pyrolysis of biomass.

### BACKGROUND

Biomass, such as forestry and agricultural products and residues, is a major underutilized product in the world. The U.S. Department of Energy estimated that there are over 12 million dry tons of biomass residues generated each year in Georgia that could be used for energy and chemical production. There are several different technologies for converting the biomass to useful energy (e.g., direct burn, co-firing, gasification, and the like) or to biobased products (e.g., fermentation, pyrolysis, and the like), in particular bio-oil. Depending on the type of process used, the final product may have different values and applications. In most cases these products replace those generated from crude oil, thus having long-term sustainability and environmental benefits (e.g., being carbon neutral).

Bio-oil is a mixture of water, light volatiles, and non-volatiles and is highly reactive because of the presence of significant quantities of oxygen. Therefore, the common method of distillation (as performed with crude oil) for separation of fractions is not effective. During distillation, the oils start boiling below 100° C., accompanied by numerous polymerization reactions, and distillation stops around 250 to 280° C. leaving as much as 50% of the starting material as residue.

The ability to cool the bio-oil from process temperatures around 450° C. or higher and simultaneously fractionate it would yield a variety of useful products. It is known from previous experience that slow condensation (e.g., condensation that takes place over a time period greater than 2 seconds) can result in reactions between compounds, thus increasing the tar fraction of the condensed bio-oil. In addition, using condensation coils provides surfaces on which tar could deposit and further enhance (catalyze) tar formation. Therefore, there is a need in the industry for an alternate method of condensing the bio-oil.

### SUMMARY

Pyrolysis systems and methods of recovering bio-oil product of are disclosed. An illustrative embodiment of a pyrolysis system, among others, includes: a pyrolysis reactor that produces a first bio-oil stream; an injection spray system for receiving the first bio-oil stream, wherein the injection spray system is configured to spray a solvent liquid onto the first bio-oil stream to produce a second bio-oil stream, wherein the injection spray system is configured to control the removal of heat from the first bio-oil stream; and a post injection spray system that is configured to receive the second bio-oil stream.

An illustrative embodiment of a method of recovering bio-oil product, among others, includes: providing a first bio-oil stream; spraying a solvent liquid onto the first bio-oil stream; removing heat from the first bio-oil stream; and recovering a second bio-oil stream.

Other systems, methods, features, and advantages of this disclosure will be or become apparent to one with skill in the art upon examination of the following drawings and detailed description. It is intended that all such additional systems, methods, features, and advantages be included within this description, be within the scope of this disclosure, and be protected by the accompanying claims.

### BRIEF DESCRIPTION OF THE DRAWINGS

Many aspects of the disclosure can be better understood with reference to the following drawings.

FIG. 1 illustrates an embodiment of a pyrolysis system.

FIG. 2 illustrates an embodiment of a flow chart to recover bio-oil using the pyrolysis system illustrated in FIG. 1.

FIG. 3 illustrates a simulation model for an embodiment of a pyrolysis system in which the solvent liquid is ethanol.

FIG. 4 illustrates a simulation model for an embodiment of a pyrolysis system in which the solvent liquid is methanol.

FIG. 5 illustrates a simulation model for an embodiment of a pyrolysis system in which the solvent liquid is acetone.

FIG. 6 illustrates a simulation model for an embodiment of a pyrolysis system in which the solvent liquid is toluene.

FIG. 7 illustrates a simulation model for an embodiment of a pyrolysis system in which the solvent liquid is acetic acid.

FIG. 8 illustrates a simulation model for an embodiment of a pyrolysis system in which the solvent liquid is water.

### DETAILED DESCRIPTION

Embodiments of the present disclosure will employ, unless otherwise indicated, techniques of chemistry, physics, and the like, which are within the skill of the art. Such techniques are explained fully in the literature.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

It must be noted that, as used in the specification and the appended claims, the singular forms “a,” “an,” and “the” include plural referents unless the context clearly dictates otherwise. Thus, for example, reference to “a support” includes a plurality of supports. In this specification and in the claims that follow, reference will be made to a number of terms that shall be defined to have the following meanings unless a contrary intention is apparent.

### DEFINITIONS

In this specification and in the claims that follow, reference will be made to a number of terms that shall be defined to have the following meanings unless a contrary intention is apparent.

“Biomass” can be created as products, by-products, and/or residues of the forestry and agriculture industries. Biomass includes, but is not limited to, forest and mill residues, agricultural crops and wastes, wood and wood wastes, animal wastes, livestock operation residues, aquatic plants, fast-growing trees and plants, and municipal and industrial wastes. In particular, biomass can include cellulose, hemicelluose, and/or lignin.

“Pyrolysis” is the thermal conversion of biomass in the absence of oxygen at temperatures around 400 to 500° C. When treated at these temperatures, biomass decomposes to three primary products, namely, charcoal, bio-oil, and gases (e.g., CO, H₂, CO₂, and CH₄).

As used herein, “bio-oil” is a mixture of water, light volatiles, and non-volatiles and is highly reactive because of the presence of significant quantities of oxygen. At temperatures around 450° C. the bio-oil is a complex mixture of chemical species that result from the decomposition of cellulose, hemicellulose, and lignin. There are over 300 compounds identified that include, but are not limited to, hydroxyaldehydes, hydroxyketones, sugars, carboxylic acids, and phenolics. The abundance of these chemical species in bio-oil makes it similar to crude petroleum oil, and thus an attractive resource for obtaining chemicals and fuels.

The following “symbols” are used in the equations described in this disclosure. The symbols are defined as:

A Spherical droplet surface area, m²;
D Diameter of spherical droplet, m;
h Convective heat transfer coefficient, W/m²-K, or enthalpy (heat) kJ/kg;
k Thermal conductivity of the liquid, W/m-K;

### m Mass, kg;

N Number of droplets;
Nu Nusselt number;
Q Energy transferred during current time step, J; [symbol]
Energy transfer rate during current time step, W;
Re Reynolds number; and
Pr Prandtl number.

The following “subscripts” are used in the equations described in this disclosure. The subscripts are defined as:

### D Diameter;

fg heat of vaporization; and
rat ratio.

### General Discussion

Pyrolysis systems and methods of use thereof, are described herein. In general, the pyrolysis system can be used to create bio-oil products in a biomass pyrolysis conversion and subsequently capture these products during a condensation process. An advantage of the pyrolysis system described herein is its ability to control (e.g., limit) the production of phenolic compounds, which can complicate the capture of more valuable bio-oil products and substantially increase the requirements for cleaning a pyrolysis system. The pyrolysis system is configured to control the rate of condensation and the temperature at which condensation occurs, as well as to provide a medium to collect the products once condensed.

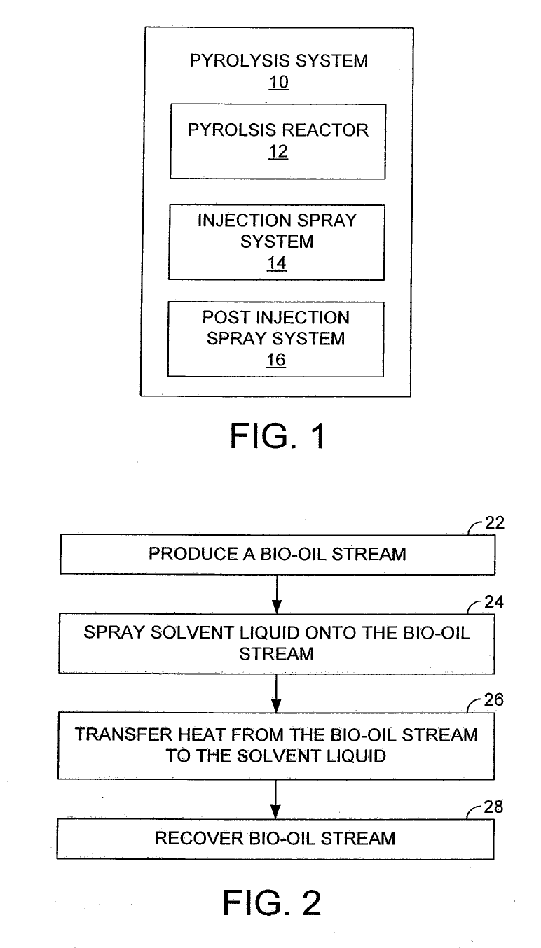

FIG. 1 illustrates a pyrolysis system 10 that includes, but is not limited to, a pyrolysis reactor 12, an injection spray system 14, and a post injection spray system 16. In gereral, a bio-mass is added to the pyrolysis reactor 12 and a bio-oil vapor is formed. The bio-oil vapor is at an elavated temperture (from about 350 to 500° C.). The bio-oil vapor is transferred to the injection spray system 14 in a bio-oil stream. For example, an inert gas (e.g., nitrogen, helium, argon, and the like) can be used to flow the bio-oil vapor into the injection spray system 14.

A solvent liquid is sprayed into the bio-oil stream and heat is removed from the bio-oil stream. In general, a substantial portion of the heat can be rapidly removed from the bio-oil stream, and the system can be designed to provide condensation in about 2 seconds or less. The liquid solvent is atomized upon ejection and the atomized solvent liquid droplets have a very large surface area to volume ratio, which allows for rapid heat transfer from the higher temperture bio-oil stream to the solvent liquid droplets. In embodiments, where the bio-oil or portions thereof are miscilbe in the solvent liquid, the bio-oil is incorporated into the solvent liquid droplets. As heat energy is transferred from the bio-oil to the solvent liquid droplet, all or a portion of the solvent liquid is evaporated. This process can occur numerous times until the bio-oil stream is cooled to a predetermined temperature range. Subsequently, the cooled bio-oil/solvent liquid stream is collected and the inert gas/solvent vapor mixture is processed in the post injection spray system 16. In an embodiment, the solvent can be condensed and re-captured for future use.

FIG. 2 illustrates a flow chart for the recovery of bio-oil using the pyrolysis system 10 illustrated in FIG. 1. In Block 22 a bio-oil stream is produced from a bio-mass. In Block 24 the solvent liquid is sprayed onto the bio-oil stream. In Block 26 the heat, or at least a portion thereof, from the bio-oil stream is transferred to the solvent liquid. In Block 28 the bio-oil is recovered. In an embodiment, the vaporized solvent from the spray cooling is re-condensed from the inert carrier gas/solvent vapor stream.

The pyrolysis reactor 12 is a chamber in which bio-mass raw materials are heated to the point at which chemical conversion of the bio-mass occurs. The process generally involves heating in an inert (in the absence of oxygen) atmosphere (e.g., nitrogen, helium, argon, and the like) to prevent combustion. As the bio-mass is heated (e.g., in a pre-determined heating ramp) and held in a predetermined tempreature range for a specified time frame, the material is broken down into simpler components that are given off as bio-oil vapors (e.g., one or more compounds). The pyrolysis reactor 12 is interfaced with the injection spray system 14. The bio-oil stream is produced in the pyrolysis reactor 12, and then the bio-oil stream is introduced into the injection spray system 14. One skilled in the art understands pyrolysis reactors 12 and how they operate, so additional details are not disclosed here. One reference that further describes pyrolysis is: Boucher (1977), Pyrolysis of industrial wastes for oil and activated carbon recovery. Environmental Protection Agency Office of Research and Development Industrial Environmental Research Laboratory, which is incorporated herein by reference.

In general, the injection spray system 14 controls the diameter of the solvent liquid droplets, the solvent mass flow rate, the method and location(s) for injecting the solvent spray, the solvent temperature in the injection spray system, and combinations thereof. The injection spray system 14 includes, but is not limited to, one or more injectors and a container, as well as other components to store, pump, and/or transfer the solvent liquid. The injectors can be positioned at one or more positions within the container. For example, an injector can be positioned adjacent the entry point of the bio-oil stream. In addition or in the alternative, an injector can be positioned downstream from the entry point of the bio-oil stream. In an embodiment, multiple injectors can be positioned at multiple points in the container. One or more of the multiple injectors can be used in tandem or individually to control one or more parameters in the injection spray system 14. For example, one or more of the injectors can be used (e.g., turned on/off, flow rate changed, diameter of the droplets adjusted, and the like) to control the rate of condensation and/or the temperature at which condensation occurs.

The container in the injection spray system 14 receives the bio-oil stream from the pyrolysis reactor 12. The injectors spray the solvent liquid into the bio-oil stream as the bio-oil stream enters the container of the injection spray system 14. The solvent liquid can be atomized into solvent liquid droplets as the solvent liquid is sprayed into the container. The injection spray system 14 can control the rate at which the solvent liquid is sparyed into the container as well as control the size of the solvent liquid droplets. The type of solvent liquid, the droplet size, and the rate at which the solvent liquid is sprayed are all parameters that influence the rate of heat transfer and thus can be used to control the rate of condensation and the temperature at which condensation occurs.

In an embodiment, a plurality of solvent liquids can be used. In an another embodiment, a plurality of droplet sizes can be sprayed into the container. In an another embodiment, a plurality of mass flow rates can be used to spray the solvent droplets into the container. In another embodiment, a plurality of solvent liquids can be used, a plurality of droplet sizes can be sprayed into the container, and/or a plurality of mass flow rates can be used to spray the solvent droplets into the container.

In an another embodiment, a control feedback system using the state of the downstream bio-oil product vapor and condensed liquid can be used to alter the solvents used, modify the droplet sizes, turn on/off one or more injectors, and/or modify the mass flow rate at which the solvent is sprayed into the container. The state of the downstream bio-oil products can include, but is not limited to, the temperature, the products being collected, and/or the bio-oil product viscosity. In another embodiment, the state of the bio-oil flowed into the injection spray system 14 can be determined and one or more parameters mentioned above can be appropriately adjusted.

The post injection spray system 16 collects the condensed bio-oil products and liquid solvent. It also further cools the product stream to condense and capture all remaining solvent vapor. It then collects the liquid bio-oil products and solvent mixture for later refining, separation, or processing into marketable products.

The solvent liquids can include, but are not limited to, alcohols (e.g., low molecular weight alcohol compounds (e.g., C₂-C₄) such as methanol, ethanol, propanol, and the like), mineral spirits, esters (e.g., alkyl acetate (e.g., ethyl acetate, butyl acetate, and the like)), aromatic hydrocarbons (e.g., toluene, organic acids (e.g., acetic acid, and the like), and the like), water, and combinations thereof (e.g., a polar compound and a non-polar compound (e.g., water and ethanol)). In an embodiment, the solvent liquid is ethanol, methanol, acetone, toluene, acetic acid, water, or combinations thereof. In another embodiment, the solvent liquid can be one of ethanol, methanol, or water. The choice of a specific embodiment would be based on the intended application of the recovered bio-oil. For example, in transportation fuel applications, ethanol may be an excellent solvent for bio-oil, and blends could be directly used in internal combustion engines.

The solvent liquid droplet diameter can range from several micrometers (10⁻⁶ m) to about 10 mm. In an embodiment, the solvent liquid spray can include solvent liquid droplets of one size or of a plurality of sizes.

The rate at which the solvent liquid is introduced to the container depends, at least in part, on the solvent liquid, solvent liquid droplet size, the composition of the bio-oil, the rate at which the bio-oil stream is introduced to the container, the rate of condensation desired, the temperature of the container, the final product viscosity, and combinations thereof.

A factor in determining the effectiveness is the ratio of mass flow of solvent liquid being introduced compared to the mass flow rate of the bio-oil vapor stream. The mass flow rate ratio can be from about 0.01 to 2.5.

### EXAMPLES

Now having described the embodiments of the pyrolysis system, in general, example 1 describes some additional embodiments of the pyrolysis system. While embodiments of pyrolysis system are described in connection with example 1 and the corresponding text and figures, there is no intent to limit embodiments of the pyrolysis system to these descriptions. On the contrary, the intent is to cover all alternatives, modifications, and equivalents included within the spirit and scope of embodiments of the present disclosure.

### Example 1

### Description

One feature in the conversion of bio-oil is control of the condensation process. The rate of condensation and the temperature at which the condensation occurs has been shown to be a factor in determining the final composition of the liquid bio-oil product. One problem encountered with the design and implementation of a biomass pyrolysis system is the creation of phenolic compounds. These materials have a tar-like consistency and make the capture and collection of bio-oil products more difficult through an increased requirement for equipment cleaning and in reducing the yield of more commercially valuable bio-oil products. The method chosen for condensing the vapors should provide rapid heat transfer capability.

The method described here is to use injection spray cooling to remove heat from the bio-oil vapor product stream coming directly out of the reactor. Injection spraying provides the rapid cooling necessary. Another concern is the actual collection of the desirable product condensate. The material selected should be miscible with the anticipated bio-oils and should not be a potential contaminant to that product. Therefore, it is proposed that a compatible material, such as a solvent, be used in the spray.

Not only does the spray cooling system provide rapid heat transfer, but the spray rate or properties of the droplets could be adjusted to control temperature, for example. Controlling the temperature(s) at which condensation occurs could provide a method of adjusting the final product composition. Research indicates that reducing the temperature of the bio-oil vapor stream to a certain level results in condensation of all bio-oil product vapors, with that temperature being approximately 200° C. Proper selection of the spray cooling injection rate, injected liquid material or mixture composition, and temperature can allow for more of the control of the bio-oil vapor product capture and condensation process. The system and method described in this example are summarized in FIGS. 1 and 2 and are discussed in detail above.

The proposed concept uses the inherently high heat transfer capability in the vaporization of the cooling liquid into the higher temperature bio-oil vapor stream to rapidly cool the vapor (Equation 1). The injected atomized liquid provides a very large surface area to volume ratio that allows for maximum and rapid heat transfer from the high temperature bio-oil vapor to the liquid. As the bio-oil vapor reaches the condensation temperature for each component species, the product vapor could condense on the surface of the droplet. If the bio-oil species is miscible with the liquid, then it will be incorporated into the liquid droplet. Since the bio-oil products will vary, it may be advantageous to use a mixture of liquids in the cooling spray, for example an alcohol and water mixture to capture both polar and non-polar compounds. As heat energy is transferred from the vapor stream to the liquid droplet, some of the liquid spray will evaporate and become part of the vapor stream. This heat transfer cools the bio-oil vapor stream.

[symbol]

(1)

where:
[symbol] the heat transfer rate for cooling of the bio-oil product vapor (kW or Btu/hr)
[symbol] mass flow of evaporated liquid (kg/sec)
h_{fg}=heat of vaporization as liquid is evaporated (material specific property) (kJ/kg)

The condensed bio-oil product and cooling liquid are collected downstream of the spray system. Some of the liquid spray will evaporate in the hot/warm vapors of the product stream. Therefore, a downstream condensation system should also be considered to reduce air pollution potential if the spray material is a pollutant, such as a volatile organic compound (VOC) such as ethanol. This also provides a way to recycle the liquid for reuse in the spray cooling system.

Selection of a material for spray cooling should be based on several criteria include, but are not limited to, the reactivity with the bio-oil products and thermal properties, such as heat of vaporization, boiling point, and specific heat. Materials to use in spray cooling might include, but not be limited to: alcohols such ethanol or methanol; mineral spirits; esters such as ethyl or butyl acetate; aromatic hydrocarbons such as toluene; organic acids such as acetic acid; or even water. Various combinations of these materials might be advantageous. For example, a combination of water and ethanol as the cooling liquid would allow for the downstream collection of both polar and non-polar compounds, which could be easily separated later downstream.

### Modeling of Liquid Spray Cooling Process

This section describes the mathematical model used to perform a simulation. The initial analytical study of the potential of liquid spray cooling considered the list of materials in Table 1.

TABLE 1
Potential Spray Cooling Liquids Studied
Potential Materials
for
Liquid Spray
Cooling
Ethanol
Methanol
Acetone
Toluene
Acetic Acid
Water

A simulation model was developed for use in Matlab, a commercially available software package from The MathWorks Company. The model tracks the bulk mean temperature of the bio-oil vapor stream and of the liquid droplets from the time of initial spray injection through a set period of time. A discrete time marching modeling approach was selected to track the heat and mass transfer as the hot bio-oil vapors cool through interaction with the liquid spray. The bio-oil vapor is assumed to enter at an initial temperature of 400° C., and all solvent liquid droplets are assumed to be the same uniform size (for simplicity in this analysis).

The model tracks the two flow stream temperatures and liquid spray mass during two different modes. The first mode is when the sprayed liquid is less than its boiling point (assumed at atmospheric pressure); in this mode the simplified model assumes only heat transfer occurs. Once the boiling point is reached, both heat and mass transfer occur between the liquid spray and the bio-oil vapor. Heat transfer is computed according to the following equation

[symbol]

NhA(Tᵥₐₚₒᵣ−T_{liquid})[W] (1)

where:

N=Total number of droplets

h=Convection heat transfer coefficient

A=Surface area of one liquid droplet

The convective heat transfer coefficient, h, is found from a definition of the Nusselt number (Nu), the droplet diameter (D), liquid thermal conductivity (k), and correlation for a liquid sphere in a gas stream.

$$Nu = \frac{hD}{k} so \text{:} h = \frac{Nu \cdot k}{D} \qquad ( 2 )$$

The liquid droplets are assumed to be spheres of uniform size for simplicity. Numerous techniques could be used to evaluate the Nusselt number, but these require knowledge of the flow velocity of bio-oil vapors across the liquid droplets. At the initial injection point of liquid spray into the bio-oil stream, there will be a flow velocity of the droplets with respect to the vapor. This velocity will rapidly decrease until the time when the droplets are moving downstream at approximately the same speed as the bio-oil vapor. The relationship between the spherical liquid droplet, the average Nusselt number based on the diameter, and flow stream conditions is taken from Incropera and DeWitt [2002], based on a falling liquid droplet.

N̅u̅_{D}=2+0.6Re_{D}^{1/2}Pr^{1/3} (3)

At the lower limit, when the droplet velocity with respect to the vapor is approximately 0, the Nusselt number correlation reduces to a value of 2. For this initial evaluation of the spray cooling concept, a constant value of N̅u̅_{D}=2 was assumed.

Once the rate of heat transfer is estimated, the total energy transferred between the vapor and the liquid droplets is computed by multiplying by the model time step (Δt).

Q= [symbol] Δt[J] (4)

This energy will heat up the droplet during the first mode of cooling, or evaporate some liquid from the droplet during the second mode after reaching the liquid boiling temperature.

During the initial mode, with the liquid spray heating up, the temperature change of the liquid during the current time step is computed from the following equation.

ΔT_{liquid}=Q/(mcₚ)_{liquid}[K] (5)

A similar equation will determine the temperature change in the bio-oil vapor, assuming no condensation of the vapor components occurs.

ΔTᵥₐₚₒᵣ=Q/(mcₚ)ᵥₐₚₒᵣ[K] (6)

The model tracks the liquid droplet bulk mean temperature until it reaches the saturation (boiling) temperature. From that point on, both heat and mass transfer occur. Heat transfer is still computed by equations 4-6, and the amount of liquid that goes to the vapor state is determined based on the heat of vaporization (h_{fg}) for that particular material.

Δm_{liquid→vapor}=Q/h_{fg}[kg/s] (7)

The vaporization of some liquid causes a corresponding decrease in the droplet size, so a new droplet diameter is computed assuming a still spherical shape. Equation 2 is then used to compute a new convective heat transfer coefficient (h), and the simulation model is ready for the next time step to occur.

The simulation proceeds until either a maximum time of 2 seconds is reached or the bio-oil vapor reaches an equilibrium temperature with the liquid. If the bio-oil vapor and liquid spray reach equilibrium, this means that the vapor is much less than the desired goal of 200° C. by the end of the critical first two seconds (since all materials evaluated had boiling points much less than 200° C.). At the completion of the simulation, a categorization of the case is completed. Various alternative situations could have occurred, and these are:
I. All the liquid evaporates before the two second target, and the bio-oil vapor never cools to 200° C.;
II. All the liquid evaporates before the two second target, and the bio-oil vapor cools to 200° C. or lower;
III. By the end of the two second target, the bio-oil vapor has not yet cooled to 200° C. but liquid still exists in the flow stream;
IV. The bio-oil is 200° C. or less but not yet at temperature equilibrium at the end of the two second target time period, and liquid still exists;
V. The liquid never gets to saturation temperature by the end of the two second target period, and the vapor never cools to 2000° C.; and
VI. The liquid never gets to saturation temperature by the end of the two second target period, but the vapor has cooled below 200° C.

Alternative end points I and II are not desirable, as no liquid remains in the stream to help collect bio-oil product. Alternative III is representative of a case where too little heat transfer area existed for the vapor to cool as desired, but sufficient liquid mass flow may exist. This represents a case where the droplets are likely too large to give effective cooling. The most desirable end point is case IV, where the bio-oil vapor has cooled below the desired level of 200° C. and liquid still remains to collect and transport the product. Alternatives V and VI represent situations where too much liquid and/or too large droplets exist such that the liquid never reaches boiling temperature, and the vapor is greater than or less than 200° C., respectively. Case V is perhaps the worst possibility, where a combination of too much liquid or too large droplet size leads to insufficient heat transfer for both the liquid and vapor. In this case, the vapor does not reach 200° C., and the liquid does not even reach the boiling point.

Since an explicit modeling approach was used, a dynamic time-step adjusting process was included to ensure that the solution process remained stable.

The study investigated the combination of varying initial mass flow and liquid spray droplet diameters, with the mass flow being based on a range of the liquid spray mass to bio-oil vapor mass ratio (mᵣₐₜ). The mass flow of the inert carrier gas (nitrogen) is included in the total bio-oil vapor mass flow rate considered. This initial feasibility study considered mass flow rate ratios ranging from 0.01 to 2.5 in combination of droplet diameters ranging from 0.0001 to 0.005 m.

### Simplifying Assumptions in the Model

Several simplifying assumptions were made in the analysis model. These include:
The detailed effects of condensation of bio-oil vapor products are not considered. The purpose of this proposed new technology is to rapidly cool the vapor to the point were condensation of products in a more desirable form occurs. During biomass pyrolysis, an inert gas such as nitrogen is used as a carrier for the bio-oil vapors produced; however the product vapors will condense.
No heat loss from the bio-oil vapor through the surround pipe or duct wall is included. Since the desire is to cool the vapor below 200° C. level within two seconds, the impact of the slower convection loss to the pipe wall will be minimal in this short time frame.
No vaporization of the liquid spray is assumed until the liquid reaches the saturation temperature. During the simulation runs discussed below, the saturation temperature was generally reached fairly rapidly, so the impact of evaporation before reaching the “boiling” temperature is considered minimal.
The potential for reaching saturation conditions of bio-oil products condensing and diffusing into the liquid droplets is not considered; again because the bio-oil vapor stream composition can vary.

### Sample Model Results

To investigate spray cooling to improve bio-oil product condensation, the simulation model was run using the list of potential materials given in Table 1.

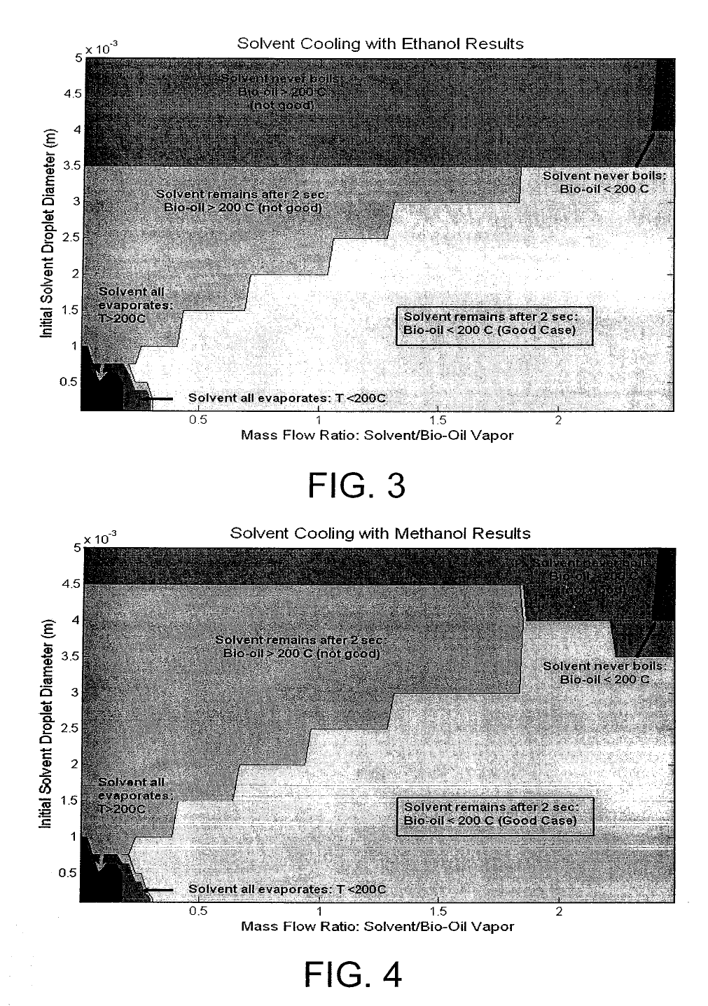

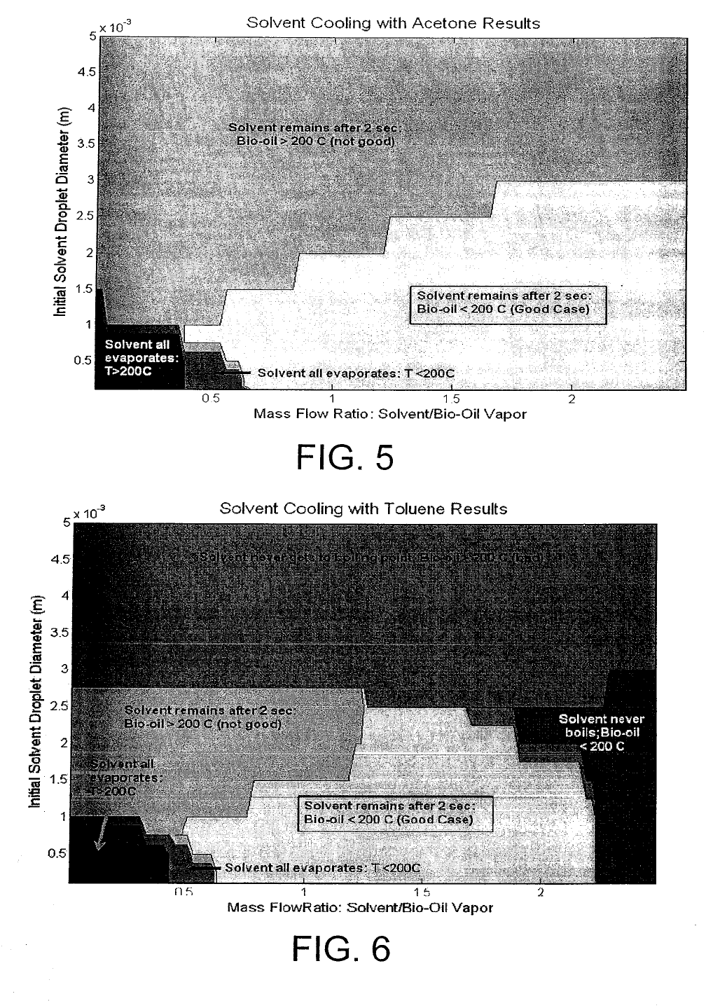

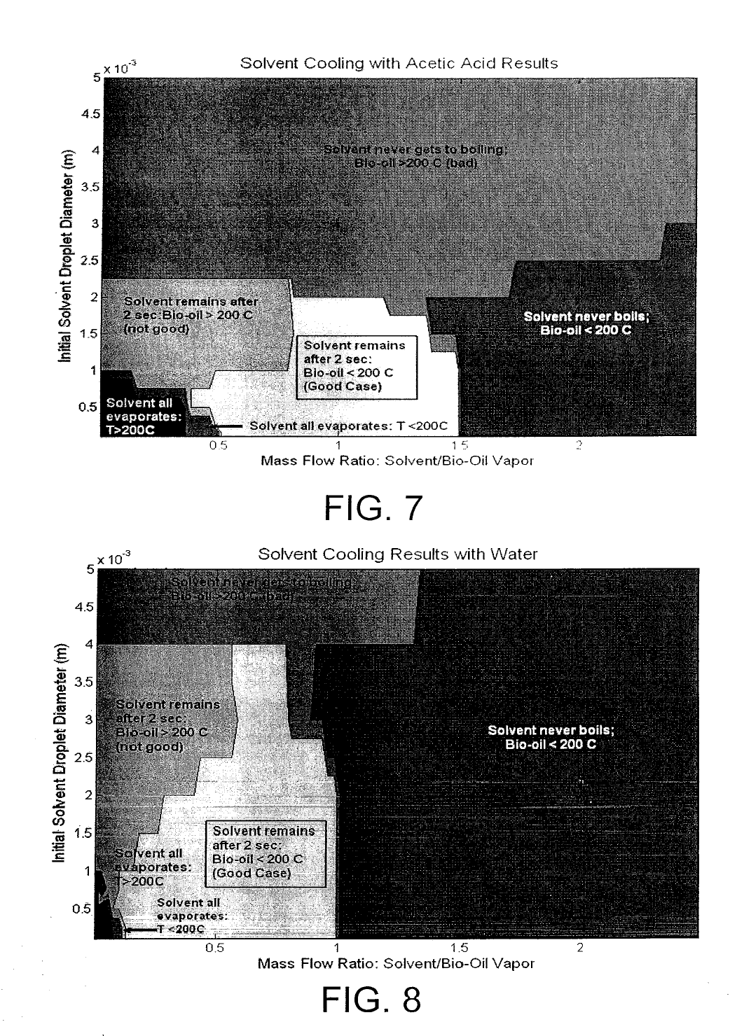

The results in this example are summarized in FIGS. 3 through 8. Particularly good results are predicted for using ethanol, methanol and water, while acceptable results could be obtained using any of the liquids studied. Each of these has a relatively high heat of vaporization. Many of the more desirable bio-oil products are easily soluble in alcohols like ethanol and methanol. A mixture of both ethanol and water may be a very desirable combination.

In general, it is believed that the liquid spray flow rate should be kept to fairly low levels. This simulation indicates that successful results can be obtained for mass flow ratios less than about 0.5 (liquid/bio-oil vapor), although a somewhat higher flow rate would be necessary for toluene and acetone. Diameters less than 0.5 mm appear to have a greater potential for all of the liquid to evaporate or to require higher mass flow rates to get the desired vapor cooling effect. Desired results are achieved with initial spray droplets in the range of 0.5 to 1.5 mm diameter and relatively low mass flow ratios (0.5 to 0.8) for all materials studied. Ultimately, the ideal combination of flow rate ratio and droplet diameter is dependent on the actual liquid material or material combination used.

### CONCLUSION

This Example describes systems and methods for rapid cooling of bio-oil vapors from a pyrolysis process. The spray injection of liquid into the bio-oil vapor stream can provide the rapid cooling needed to minimize production of undesirable tar-like phenolic compounds. The presence of liquid in the product stream may facilitate downstream collection of the desirable products. Proper selection of the liquid spray injection rate may allow for control of the temperature levels where condensation occurs, thus potentially giving the ability to control or at least influence the product composition mix.

Results of a heat and mass transfer simulation model indicate that the embodiments modeled work well in achieving the desired rapid cooling affect, which is to cool the bio-oil vapors to below about 200° C. within about two seconds after leaving the pyrolysis chamber.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range.

Many variations and modifications may be made to the above-described embodiments. All such modifications and variations are intended to be included herein within the scope of this disclosure and protected by the following claims.

We claim:

1. A pyrolysis system, comprising:
a pyrolysis reactor that produces a first bio-oil stream;
an injection spray system for receiving the first bio-oil stream, wherein the injection spray system is configured to spray a solvent liquid onto the first bio-oil stream to produce a second bio-oil stream, wherein the injection spray system is configured to control the removal of heat from the first bio-oil stream; and
a post injection spray system that is configured to receive the second bio-oil stream.

2. The pyrolysis system of claim 1, wherein the solvent liquid is selected from one of the following: alcohols, mineral spirits, esters, aromatic hydrocarbons, water, and combinations thereof.

3. The pyrolysis system of claim 2, wherein the solvent liquid is an alcohol.

4. The pyrolysis system of claim 3, wherein the alcohol is selected from methanol, ethanol, and combinations thereof.

5. The pyrolysis system of claim 2, wherein the solvent liquid is an aromatic hydrocarbon.

6. The pyrolysis system of claim 5, wherein the aromatic hydrocarbon is selected from toluene, acetic acid, and combinations thereof.

7. The pyrolysis system of claim 2, wherein the solvent liquid is water.

8. The pyrolysis system of claim 2, wherein the solvent liquid is a mixture of an alcohol and water.

9. The pyrolysis system of claim 2, wherein the solvent liquid is a mixture of a polar compound and a non-polar compound.

10. The pyrolysis system of claim 1, wherein the injection spray system is configured to atomize the solvent liquid when the solvent liquid is sprayed onto the first bio-oil product.

11. The pyrolysis system of claim 1, wherein the injection spray system is configured to control properties selected from one of the following: a diameter of the solvent liquid droplets, a mass flow rate, a mass flow rate ratio, a temperature in the injection spray system, and combinations thereof.

12. The pyrolysis system of claim 11, wherein the mass flow rate ratio is about 0.01 to 2.5.

13. The pyrolysis system of claim 1, wherein the injection spray system is configured to spray solvent liquid droplets having a diameter of about 0.1 to 5 mm.

14. A method of recovering a bio-oil product, comprising:
providing a first bio-oil stream;
spraying a solvent liquid onto the first bio-oil stream;
removing heat from the first bio-oil stream; and
recovering a second bio-oil stream.

15. The method of claim 14, wherein the solvent liquid is atomized when spraying the solvent liquid onto the bio-oil stream.

16. The method of claim 14, wherein spraying includes spraying the solvent liquid that is selected from one of the following: alcohols, mineral spirits, esters, aromatic hydrocarbons, water, and combinations thereof.

17. The method of claim 14, wherein spraying includes spraying the solvent liquid that is an alcohol.

18. The method of claim 17, wherein the alcohol is selected from methanol, ethanol, and combinations thereof.

19. The method of claim 14, wherein spraying includes spraying the solvent liquid that is an aromatic hydrocarbon.

20. The method of claim 19, wherein the aromatic hydrocarbon is selected from toluene, acetic acid, and combinations thereof.

21. The method of claim 14, wherein spraying includes spraying the solvent liquid that is water.

22. The method of claim 14, wherein spraying includes spraying the solvent liquid that is a mixture of an alcohol and water.

23. The method of claim 14, wherein spraying includes spraying the solvent liquid that is a mixture of a polar compound and a non-polar compound.

24. The method of claim 14, further comprising: controling a rate of condensation of bio-oils in the first bio-oil stream.

25. The method of claim 14, Wherein the spraying and removing heat occur in an injection spray system that controls properties selected from one of the following: a diameter of the solvent liquid droplets, a mass flow rate, a mass flow rate ratio, a temperature in the injection spray system, and combinations thereof.

26. The method of claim 25, wherein the mass flow rate ratio is from about 0.01 to 2.5.

27. The method of claim 25, wherein the diameter of the solvent liquid droplets is about 0.1 to 5 mm.

28. The method of claim 14, further comprising: reducing the temperature of the first bio-oil stream to less than about 200° C.

29. The method of claim 14, further comprising: reducing the temperature of the first bio-oil stream to less than about 200° C. in less than about 2 seconds.

30. The method of claim 14, further comprising: condensing bio-oils in the first bio-oil stream in less than about 2 seconds.

31. The method of claim 14, further comprising: recovering the solvent liquid.
